(19) United States
(12) Patent Application Publication
(10) Pub. No.: US 2024/0051920 A1
(43) Pub. Date: Feb. 15, 2024

(54) AN IMPROVED HIGHLY EFFICIENT PROCESS FOR THE PREPARATION OF NINTEDANIB AND PHARMACEUTICALLY ACCEPTABLE SALT THEREOF

(71) Applicant: BDR LIFESCIENCES PRIVATE LIMITED, Vadodara (IN)

(72) Inventors: Shah Dharmesh Mahendrabhai, Mumbai (IN); Mehta Tushar Bharatkumar, Vadodara (IN); Chavda Rajendrakumar Gokalbhai, Vadodara (IN); Kathrotiya Harshad Ghanshyambhai, Vadodara (IN); Patel Arpit Kiritbhai, Vadodara (IN); Karad Manish Bhikhabhai, Vadodara (IN); Kothadiya Sagar Premjibhai, Vadodara (IN)

(21) Appl. No.: 18/040,868

(22) PCT Filed: Aug. 6, 2021
(86) PCT No.: PCT/IN2021/050754

§ 371 Date: Feb. 7, 2023

(30) Foreign Application Priority Data
Aug. 7, 2020 (IN) 202021033796

(51) Int. Cl. C07D 209/34

(52) U.S. Cl. CPC C07D 209/34; C07B 2200/13

(57) ABSTRACT

The present invention relates to an improved highly efficient and economic process for large-scale production of Nintedanib and pharmaceutically acceptable salt thereof. The present invention also relates to a single step process that form highly pure Nintedanib through novel intermediates. In this process, Nintedanib base [I] is prepared in a single step, in-situ process wherein the process is performed by formation of two novel intermediates namely, methyl-1-(bromoacetyl)-2-oxo-2,3-dihydro-1H-indole-6-carboxylate and methyl-(3Z)-1-(bromoacetyl)-3-[methoxy(phenyl)methylidene]-2-oxo-2,3-dihydro-1H-indole-6-carboxylate. This process avoids use of expensive and hazardous reagent and solvent such as methyl cyclohexane. Further, there is no isolation and analysis of any intermediate after every step completion that made the process easy to perform without much hurdles. Along with the ease of performance, present invention process also gives high-purity final product with high yield. This makes the process highly cost-effective and time-efficient.

### FIELD OF THE INVENTION

The present invention relates to an improved highly efficient, and economic process for large-scale industrial production of Nintedanib and pharmaceutically acceptable salt thereof. The present invention also relates to preparation of highly pure Nintedanib through novel intermediates.

### BACKGROUND OF THE INVENTION

Nintedanib is a small molecule kinase inhibitor used as oral medication for the treatment of idiopathic pulmonary fibrosis and along with other medications for some types of non-small-cell lung cancer (NSCLC) and systemic sclerosis-associated interstitial lung disease. It is a small molecule tyrosine-kinase inhibitor, targeting vascular endothelial growth factor receptor, fibroblast growth factor receptor and platelet derived growth factor receptor.

Within the spectrum of idiopathic pulmonary fibrosis treatment options, Nintedanib is currently one of only two disease-modifying therapies available and indicated for the condition (the other being pirfenidone) and as such is used as a first-line treatment following diagnosis to slow down the progressive loss of lung function. As a chemotherapeutic agent for NSCLC, Nintedanib, in combination with docetaxel, is reserved for patients who have tried and failed first-line chemotherapeutic options.

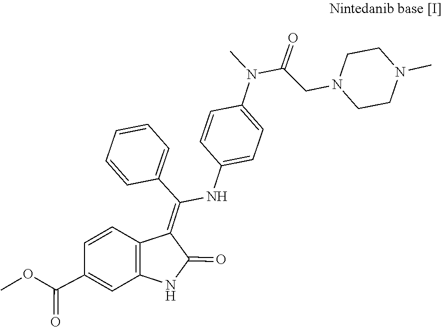

Nintedanib is having the CAS NO. 656247-17-5, chemical formula C₃₁H₃₃N₅O₄ and molecular weight 539.6248. Nintedanib is chemically known as methyl (3Z)-3-[({4-[N-methyl-2-(4-methylpiperazin-1-yl)acetamido]phenyl}amino)(phenyl)methylidene]-2-oxo-2,3-dihydro-1H-indole-6-carboxylate and structurally represented as above.

The drug is used in form of its salt with ethane sulfonic acid. This salt, Nintedanib Esylate, is a yellow, crystalline solid that melts at 244° C. to 251° C.

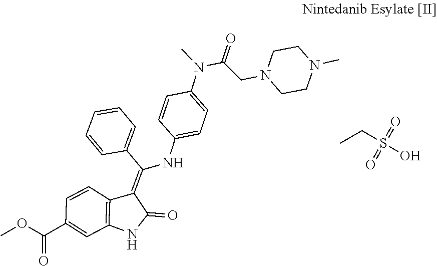

Nintedanib Esylate is the presently marketed salt in the formulation having brand name OFEV® which is developed by Boehringer Ingelheim, available in multiple strengths for oral administration capsule, which was first approved by the USFDA on Oct. 15, 2014. In the US, primarily, it was approved by the USFDA for the treatment of the treatment of idiopathic pulmonary fibrosis (IPF) and to slow declining pulmonary function in patients with systemic sclerosis-associated interstitial lung disease. Nintedanib was granted orphan drug designation in the US by the FDA for the treatment of idiopathic pulmonary fibrosis in June 2011 until 15 Oct. 2021. In March 2020, it was approved for use in the United States to treat chronic fibrosing (scarring) interstitial lung diseases (ILD) with a progressive phenotype (trait). It is the first treatment, for this group of fibrosing lung diseases that worsen over time. In the EU, Nintedanib is indicated in combination with docetaxel for the treatment of adult patients with metastatic, locally advanced, or locally recurrent non-small cell lung cancer of adenocarcinoma histology who have already tried first-line therapy. Nintedanib was first disclosed in U.S. Pat. No. 6,762,180 patent assigned to Boehringer Ingelheim. The process discloses the reaction of methyl (Z)-1-acetyl-3-(ethoxyphenylmethylene)-oxindole-6-carboxylate with N-(4-aminophenyl)-N,4-dimethyl-1-piperazine acetamide in dimethylformamide, followed by the treatment with piperidine to obtain Nintedanib free base. The use of dimethyl formamide and piperidine in this reaction make the handling and work-up difficult. The patent further discloses the use of other organic bases such as trimethylamine, N-ethyl-diisopropylamine, butylamine, dimethylamine and piperidine for this reaction, which are quite costly. Further, the yield of this reaction step is not mentioned.

WO2009/071523 patent discloses the process of preparation of Nintedanib base and its monoethanesulfonate formation. The general synthesis scheme is as below:

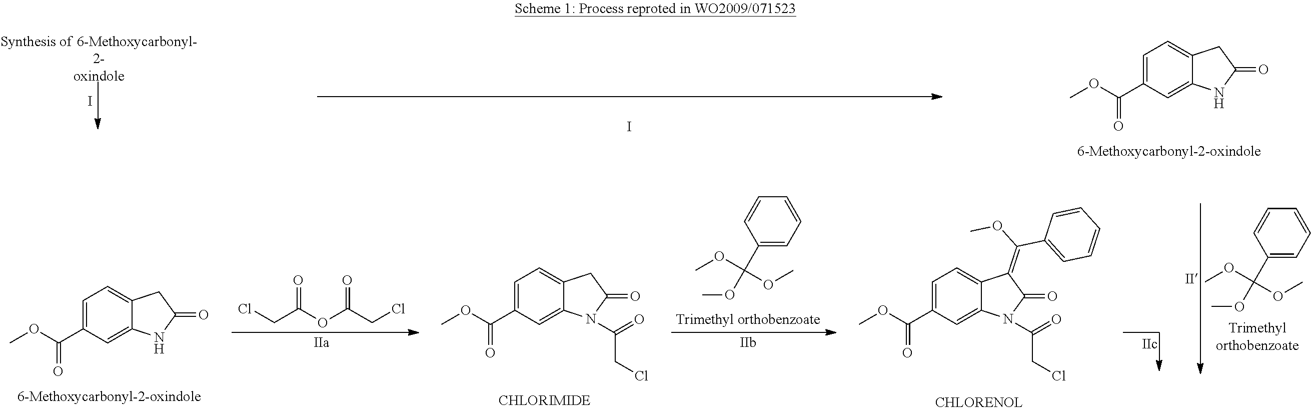

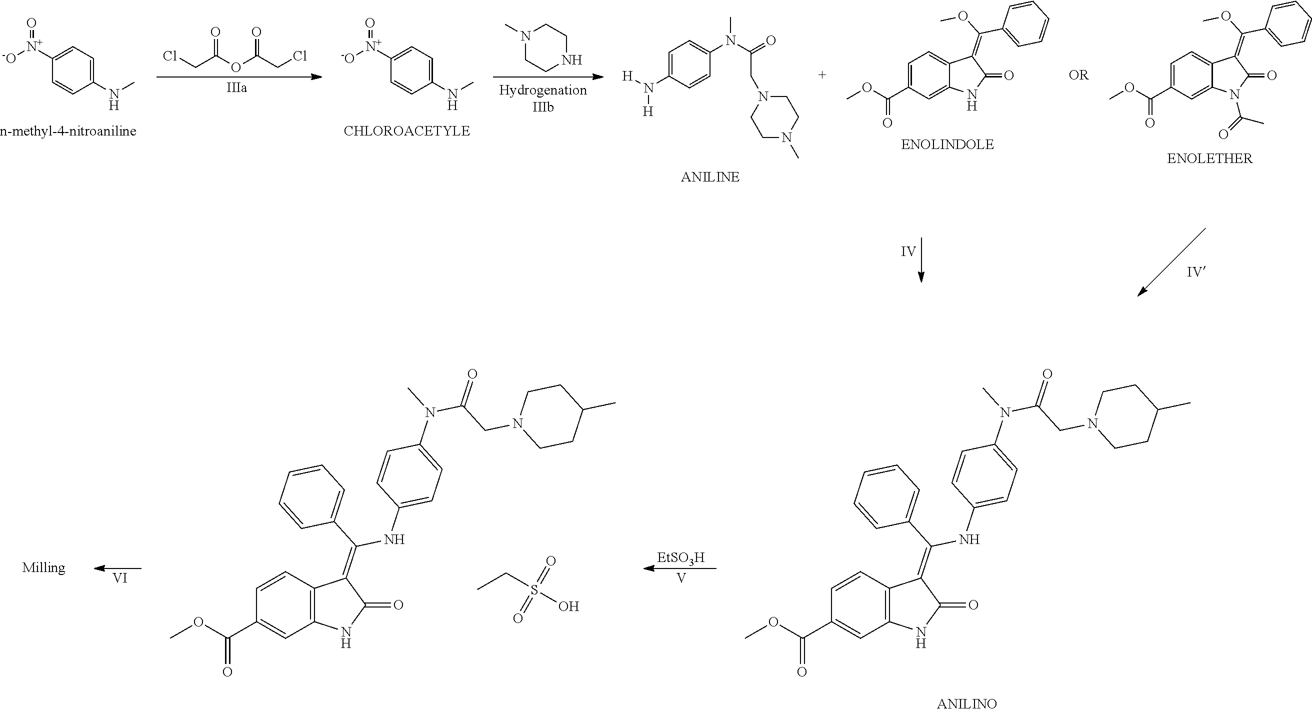

Scheme-1 discloses the preparation of Nintedanib by the condensation of methyl 2-oxo-2,3-dihydro-1H-indole-6-carboxylate with chloroacetic anhydride in presence of toluene and isolating intermediates in each step. This is important to note that by above process final product yield is less than 71.63%. Further, chloroacetic anhydride as the acylating agent is used which is very costly and have less commercial availability. Further, a considerable disadvantage of the above mentioned synthesis is the formation of the toxic methyl chloroacetate as a side product.

WO2017/016530 assigned to Zentiva discloses the use of alkali hydroxides such as potassium hydroxide; and alkali alkoxides such as potassium tert-butoxide and sodium ethoxide; for the reaction of methyl (Z)-1-acetyl-3-(ethoxyphenylmethylene)-oxindole-6-carboxylate with N-(4-aminophenyl)-N,4-dimethyl-1-piperazine acetamide to obtain Nintedanib. This process involves many steps wherein isolation of intermediates occurred using many solvents and reagents.

Above patents have long procedure steps to follow as they involve many separate steps that made them not suitable for synthesis of API in large amount industrial scale. Further, there is no mentioning of purity of the final product and each stage intermediates. So, there may be possibility towards low purity final product development. Further, isolation and purification of each stage product may lead to high cost and increase analysis steps as well as time consumption. Hence, above processes using above-mentioned reagents and solvents may produce high-cost product. Innovator is using costly solvent (methyl cyclohexane) for isolation. Further, innovator process is preparing Nintedanib base in two different stages, so relatively time consuming.

So, although the above-mentioned patent applications already describe processes for manufacture of Nintedanib and its monoethanesulfonate, there is emerging need for new and improved process for manufacture of Nintedanib that is cost effective with high purity and less time consuming.

The inventors of the present invention have developed a new and improved process for manufacture of Nintedanib base that is a single step process and use of expensive and hazardous reagent such as methyl cyclohexane is avoided. This process uses very less number of quite cheap solvents and reagents that made the process highly cost-effective and eco-friendly. Further, there is no isolation and analysis of any intermediate after every step completion that made the process less complex and easy to perform without much hurdles and procedures. Along with the ease of performance, present invention process also gives high-purity final product with high yield.

Hence, an improved process in accordance with the present invention presents above remarkable advantages when compared to the processes already described in the prior arts that made the present process highly suitable for large scale industrial production.

### OBJECTIVE OF THE INVENTION

The principal objective of present invention is to provide a single step industrially advantageous and cost-effective process for the synthesis of Nintedanib and pharmaceutically acceptable salts thereof.

Another object of the present invention is to provide an improved process for the preparation of Nintedanib which in a single step process that form highly pure Nintedanib through novel intermediates.

Another object of the present invention is to provide a cost-effective process for the preparation of Nintedanib which avoids the use of expensive reagent such as methyl cyclohexane.

Yet another object of present invention is to provide an improved efficient process for the preparation of Nintedanib and salt thereof which yields final product with high purity in high yield.

Another object of the present invention is to provide a eco-friendly process for the preparation of Nintedanib which uses very less number of solvent and reagents that are easily commercially available.

Another object of the present invention is to provide a time saving process for the preparation of Nintedanib which as it is performed in single step only without isolation of any intermediate.

One more object of the present invention is to provide a process for the preparation of Nintedanib wherein each intermediate forming during the process can be isolated also via various techniques in the prior art.

Another object of the present invention is to provide large-scale advantageous process for the preparation of Nintedanib to produce high yield final product with less complex and easy to perform single step without much hurdles and procedures.

One more object of the present invention is to provide two novel intermediates namely methyl 1-(bromoacetyl)-2-oxo-2, 3-dihydro-1H-indole-6-carboxylate [V] and methyl (3Z)-1-(bromoacetyl)-3-[methoxy(phenyl)methylidene]-2-oxo-2, 3-dihydro-1H-indole-6-carboxylate [VII].

Another object of the present invention is to provide two novel polymorph of Nintedanib Esylate namely, BDR-NIN-1 and BDR-NIN-2.

### SUMMARY OF THE INVENTION

The present invention discloses to an improved, highly efficient and economic process for large-scale industrial production of Nintedanib and pharmaceutically acceptable salt thereof. The present invention process is a single step process that form highly pure Nintedanib through novel intermediates with high yield.

One aspect of the present invention relates to preparation of Nintedanib base comprising the following reaction in a single step only:
a) methyl-2-oxo-2, 3-dihydro-1H-indole-6-carboxylate [III] is condensed with bromoacetyl bromide [IV] in presence of suitable solvent and reagents that leads to formation of a novel intermediate methyl 1-(bromoacetyl)-2-oxo-2, 3-dihydro-1H-indole-6-carboxylate [V];
b) methyl 1-(bromoacetyl)-2-oxo-2, 3-dihydro-1H-indole-6-carboxylate [V] is reacted with trimethyl orthobenzoate [VI] in presence of suitable solvent and reagent that leads to formation of another novel intermediate methyl (3Z)-1-(bromoacetyl)-3-[methoxy(phenyl)methylidene]-2-oxo-2, 3-dihydro-1H-indole-6-carboxylate [VII];
c) novel intermediate methyl (3Z)-1-(bromoacetyl)-3-[methoxy(phenyl)methylidene]-2-oxo-2, 3-dihydro-1H-indole-6-carboxylate [VII] is reacted with N-(4-aminophenyl)-N-methyl-2-(4-methylpiperazin-1-yl)acetamide [VIII] to form Nintedanib base [I].

The present invention also provides novel forms of Nintedanib Esylate designated as Form BDR-NIN-1 and Form BDR-NIN-2.

In another aspect, the present invention provides novel form of Nintedanib Esylate designated as Form BDR-NIN-1, characterized by X-ray powder diffraction (XRD) pattern having peaks at about 13.03, 17.80, 21.26 and 24.05±0.20 degrees 2-theta.

In another aspect, the present invention provides novel form of Nintedanib Esylate designated as Form BDR-NIN-2, characterized by X-ray powder diffraction pattern having peaks at about 6.54, 16.71, 18.81, 20.01, 23.19±0.20 degrees 2-theta.

In another aspect of the present invention relates to preparation of crystalline form BDR-NIN-1 of Nintedanib Esylate comprising addition of n-Heptane to crystallize out the crystalline form BDR-NIN-1.

In another aspect of the present invention relates to preparation of crystalline form BDR-NIN-2 of Nintedanib Esylate comprising addition of Methyl ethyl ketone to crystallize out the crystalline form BDR-NIN-2.

### BRIEF DESCRIPTION OF THE DRAWINGS

FIG. 1 shows an X-ray powder diffractogram of novel form of Nintedanib Esylate BDR-NIN-1.

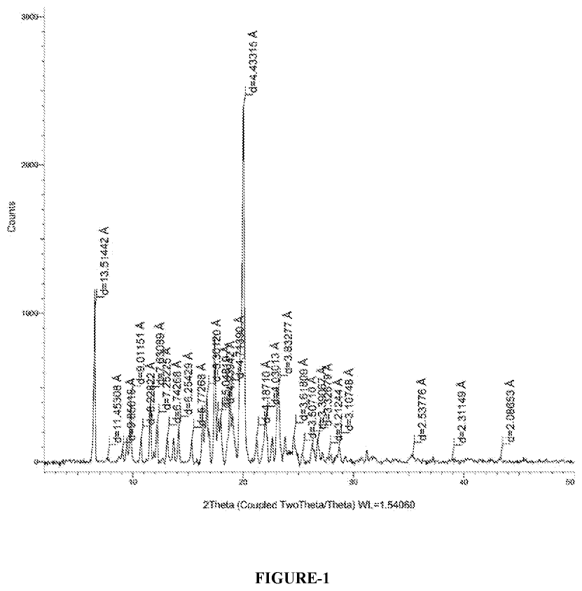

FIG. 2 shows an X-ray powder diffractogram of novel form of Nintedanib Esylate BDR-NIN-2.

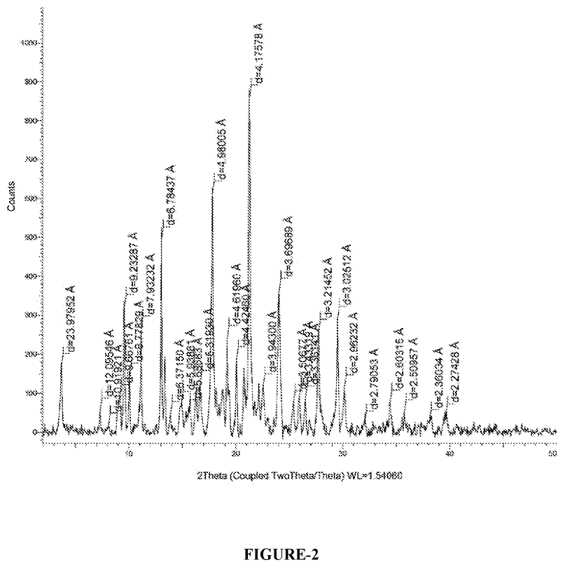

### DETAILED DESCRIPTION OF THE INVENTION

Accordingly, the present invention relates to an improved, economic and eco-friendly process for the preparation of Nintedanib that is highly suitable for large-scale industrial production of Nintedanib or pharmaceutically acceptable salt thereof.

One embodiment of the present invention provides a process for the preparation of Nintedanib wherein Nintedanib base [I] is formed in a single step process that form highly pure Nintedanib through novel intermediates.

In one embodiment of the present invention, novel intermediate methyl 1-(bromoacetyl)-2-oxo-2, 3-dihydro-1H-indole-6-carboxylate [V] is disclosed that is prepared by the condensation of methyl-2-oxo-2, 3-dihydro-1H-indole-6-carboxylate [III] with bromoacetyl bromide [IV] in appropriate solvents and reagents.

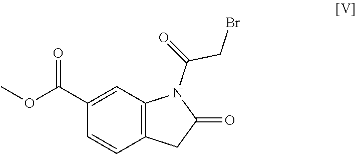 [SMILES: COC(=O)c1ccc2c(c1)N(C(=O)CBr)C(=O)C2]

In another embodiment of the present invention, novel intermediate methyl (3Z)-1-(bromoacetyl)-3-[methoxy(phenyl)methylidene]-2-oxo-2,3-dihydro-1H-indole-6-carboxylate [VII] is disclosed and prepared by the condensation of methyl 1-(bromoacetyl)-2-oxo-2, 3-dihydro-1H-indole-6-carboxylate [V] with trimethyl orthobenzoate [VI] in suitable solvent and reagent.

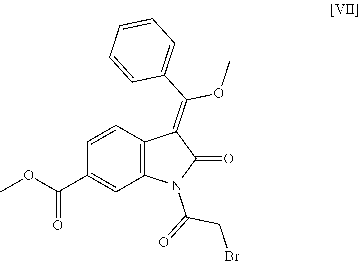 [SMILES: COC(=O)c1ccc2c(c1)N(C(=O)CBr)C(=O)/C2=C(\OC)c1ccccc1]

In one more embodiment of the present invention, Nintedanib base [I] is formed through the condensation of novel intermediate methyl (3Z)-1-(bromoacetyl)-3-[methoxy(phenyl)methylidene]-2-oxo-2, 3-dihydro-1H-indole-6-carboxylate [VII] with N-(4-aminophenyl)-N-methyl-2-(4-methylpiperazin-1-yl)acetamide [VIII] in suitable solvents and reagents.

Yet another embodiment of the present invention provides a process for the preparation of Nintedanib Esylate of formula [II] by reacting Nintedanib of formula [I] with ethanesulfonic acid using appropriate reagents and solvents.

One of the most preferred embodiment of the present invention relates to preparation of Nintedanib base [I] comprising the following reaction in a single step only:
a) methyl-2-oxo-2,3-dihydro-1H-indole-6-carboxylate [III] is condensed with bromoacetyl bromide [IV] in presence of suitable solvent and reagents that leads to provide a novel intermediate methyl 1-(bromoacetyl)-2-oxo-2, 3-dihydro-1H-indole-6-carboxylate [V];
b) methyl 1-(bromoacetyl)-2-oxo-2, 3-dihydro-1H-indole-6-carboxylate [V] is reacted with trimethyl orthobenzoate [VI] in presence of suitable solvent and reagent that leads to provide another novel intermediate methyl (3Z)-1-(bromoacetyl)-3-[methoxy(phenyl)methylidene]-2-oxo-2,3-dihydro-1H-indole-6-carboxylate [VII];
c) novel intermediate methyl (3Z)-1-(bromoacetyl)-3-[methoxy(phenyl)methylidene]-2-oxo-2,3-dihydro-1H-indole-6-carboxylate [VII] is reacted with N-(4-aminophenyl)-N-methyl-2-(4-methylpiperazin-1-yl)acetamide [VIII] to form Nintedanib base [I].

In another embodiment of the present invention, Nintedanib base [I] is further converted into Nintedanib Esylate salt [II], wherein Nintedanib base [I] is reacted with ethane sulfonic acid in suitable solvents.

The process of the present invention may be depicted as a whole in below scheme-2.

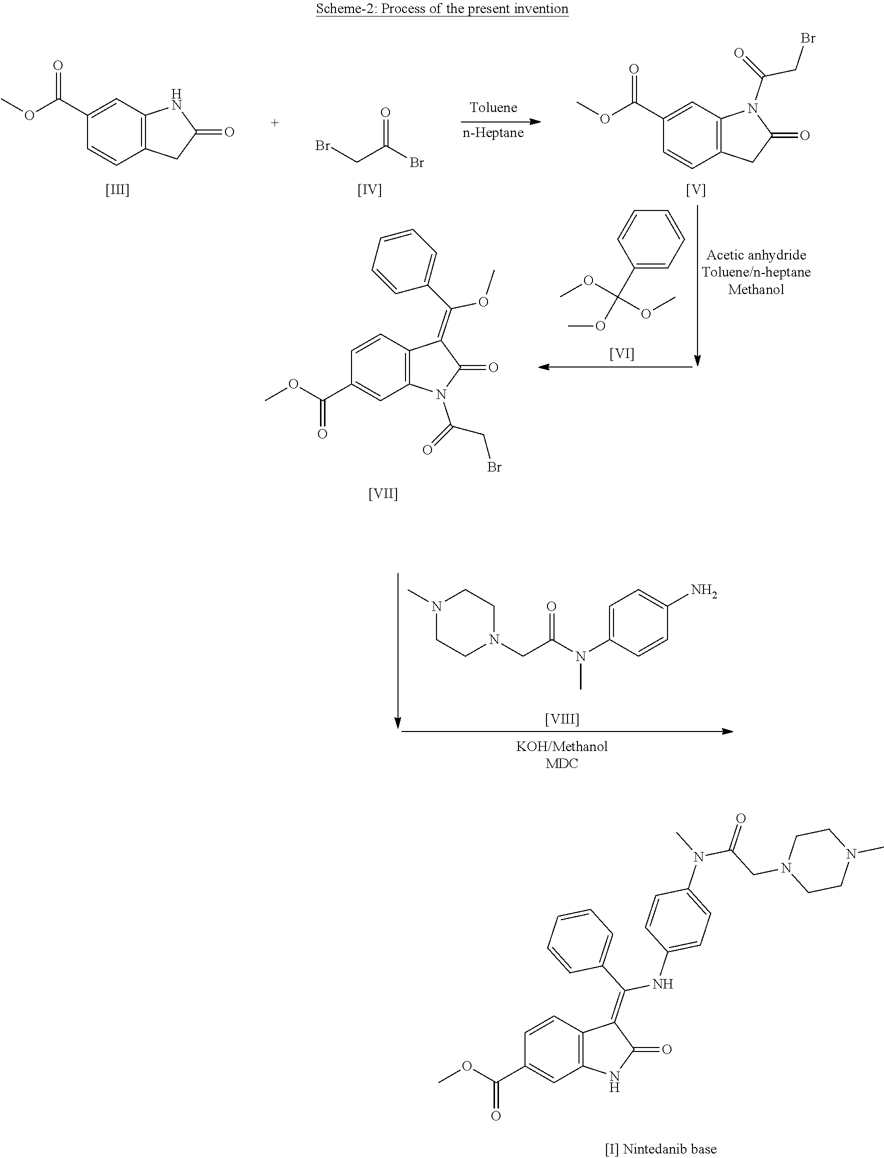

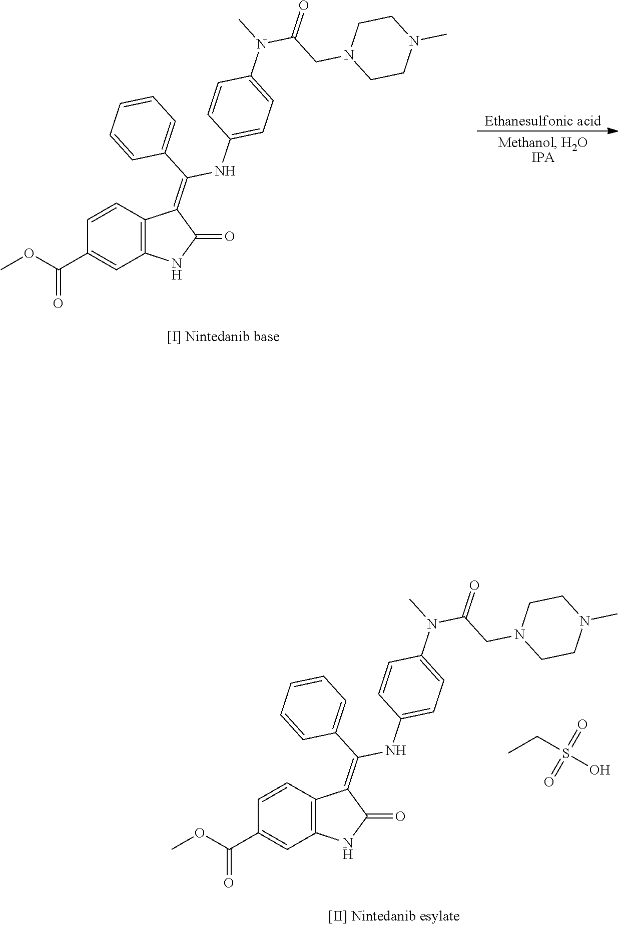

In one embodiment of the present invention provides novel form of Nintedanib Esylate designated as Form BDR-NIN-1, characterized by X-ray powder diffraction (XRD) pattern having peaks at about 13.03, 17.80, 21.26 and 24.05±0.20 degrees 2-theta. This crystalline form has better physicochemical parameters than prior-art polymorphs which includes lower hygroscopic and good flow property.

In another embodiment of the present invention provides novel form of Nintedanib Esylate designated as Form BDR-NIN-2, characterized by X-ray powder diffraction pattern having peaks at about 6.54, 16.71, 18.81, 20.01, 23.19±0.20 degrees 2-theta. This crystalline form also has better physicochemical parameters than prior-art polymorphs which includes lower hygroscopic and good flow property.

In the present invention process, Nintedanib base [I] is prepared in a single step, in-situ process, that form pure Nintedanib base with high yield. This process of present invention eliminates isolation of any intermediate that avoids unnecessary isolation and analysis steps of intermediates purification. However, isolation of all intermediates forming during the process, can be isolated via various techniques reported in the prior art. This process is time-efficient as well as cost-effective as uses cheap and easily available solvents and reagents.

One embodiments of the present invention involves preparation of Nintedanib base through formation of novel intermediates. First novel intermediate of the process is methyl 1-(bromoacetyl)-2-oxo-2,3-dihydro-1H-indole-6-carboxylate [V]. This intermediate is prepared by condensation of methyl-2-oxo-2, 3-dihydro-1H-indole-6-carboxylate [III] with bromoacetyl bromide [IV]. In this process, suitable solvent preferably toluene and methyl-2-oxo-2,3-dihydro-1H-indole-6-carboxylate [III] are charged into round-bottom flask at room temperature. Then, the reaction mixture is stirred, heated to 105-110° C. After that, bromoacetyl bromide [IV] is added slowly into the reaction mass in 60 mins at 105-110° C. The reaction mass is maintained for 8 hr at 105-110° C. Progress of reaction is monitored by TLC. After stopping the heating, the reaction mass is cooled to 60-65° C. Then, the solvent is distilled out under vacuum below 65° C. completely. Toluene is used for distilled out completely traces of bromoacetyl bromide. Then, n-heptane is added into reaction mass at 60-65° C. The reaction mixture is cooled to 10-15° C. Then, the reaction mixture is stirred for 3.0 hr at 10-15° C. The reaction mass is filtered and wash with mixture of toluene and n-heptane (twice) to get wet material of intermediate methyl 1-(bromoacetyl)-2-oxo-2, 3-dihydro-1H-indole-6-carboxylate [V].

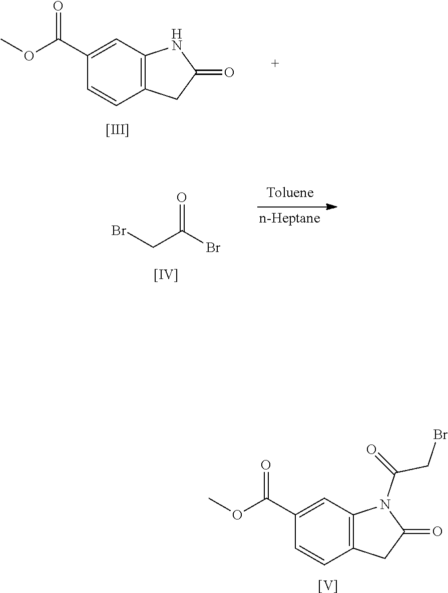

Another embodiment of the present invention involves further reaction of above compound of formula [V] that leads to preparation of another novel intermediate methyl (3Z)-1-(bromoacetyl)-3-[methoxy(phenyl)methylidene]-2-oxo-2,3-dihydro-1H-indole-6-carboxylate [VII]. In this process, clean and dry glass assembly is set in downward distillation mode. Above prepared wet material of compound [V] with suitable solvents selected preferably from toluene and acetic anhydride are charged into glass assembly at 25-30° C. The reaction mass is stirred for 10-15 minutes at 25-30° C. to get homogeneous reaction mass. The reaction mass is heated at 105-110° C. Then after, trimethyl orthobenzoate [VI] is added slowly into reaction mixture at 105-110° C. in 60 minutes. The reaction mass is maintained for 4 hr at same temperature. Volatile by product will be removed during reaction maintaining. Progress of reaction is monitored by TLC. The reaction mass is cooled to 55-60° C. Then, n-heptane is added into reaction mass at 55-60° C. The reaction mass is stirred for 15-20 minutes and then cool the reaction mixture to 10-15° C. and stirred for 2.0 hr at same temperature. The reaction mixture is filtered and washed with mixture of toluene and ethyl acetate (1:1). Obtained wet material is charged into another clean glass assembly. Methanol is added and reaction mass is stirred for 15 minutes at 25-30° C. The reaction mass is cooled at 0-5° C. and stirred for 2 hr at same temperature. Again, the reaction mass is filtered and washed with pre-chilled methanol to get wet material of compound [VII].

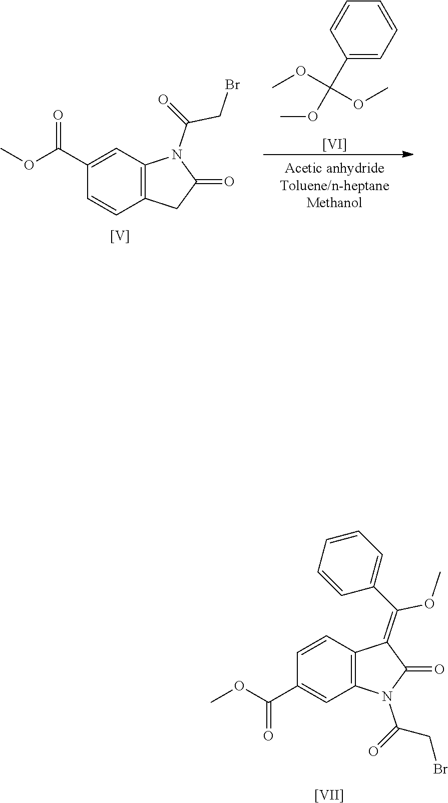

One more embodiment of the present invention involves formation of Nintedanib base of formula [I]. In this process, wet material of above prepared compound [VII] and suitable solvent selected from methanol, ethanol, propanol, and like that alcoholic solvent are charged into glass assembly. Heat the reaction mass to 60-65° C. KOH solution (in methanol) is added slowly to reaction mass at 60-65° C. in 15-20 minutes. The reaction mass is maintained for 1 hr at 60-65° C. Progress of reaction is monitored by TLC. Distill out methanol completely under vacuum at below 60° C. Again, methanol is distilled out completely under vacuum to remove traces of bromoacetic acid. Afterwards, N-(4-aminophenyl)-N-methyl-2-(4-methylpiperazin-1-yl)acetamide [VIII] is added into reaction mass at 60° C. The reaction mixture is maintained at 60-65° C. for 4 hr. Conversion is checked by TLC till completion of the reaction. After stopping the heating, the reaction mass is cooled to 10-15° C. After stirring the reaction mass for 3 hr at 10-15° C., the precipitated solid is filtered and wash with pre-chilled methanol. The reaction mass is stirred for 1 hr at 60-65° C. The reaction mixture is then cooled to 25-30° C. and stir the reaction for 2 hr at same temperature. The reaction mass is again filtered followed by wash with pre-chilled methanol. Unload wet cake and charged into another clean glass assembly. Then, methanol and dichloromethane are charged into assembly at room temperature to get clear solution. Activated charcoal is added into clear reaction mass and stir for 1 hr at 25-30° C. The reaction mass is filtered through celite hyflo bed followed by washing with mixture of methanol and dichloromethane (1:1). Dichloromethane is distilled out from filtrate ML at 40-55° C. Then again, methanol is added into reaction mass at 50-55° C. The reaction mixture is cooled to 25-30° C. and stir for 3 hr at same temperature. The reaction mass is filtered and wash with pre-chilled methanol to get wet material. The wet cake is dried under vacuum at 45-50° C. to get pure (Z)-methyl3-(((4-(N-methyl-2-(4-methylpiperazin-1-yl)acetamido)phenyl)amino)(phenyl)methylene)-2-oxoindoline-6-carboxylate (Nintedanib base) (I) as bright yellow solid. The yield of this step is 80.14% (KSM to Base formation) and purity is 99.79%.

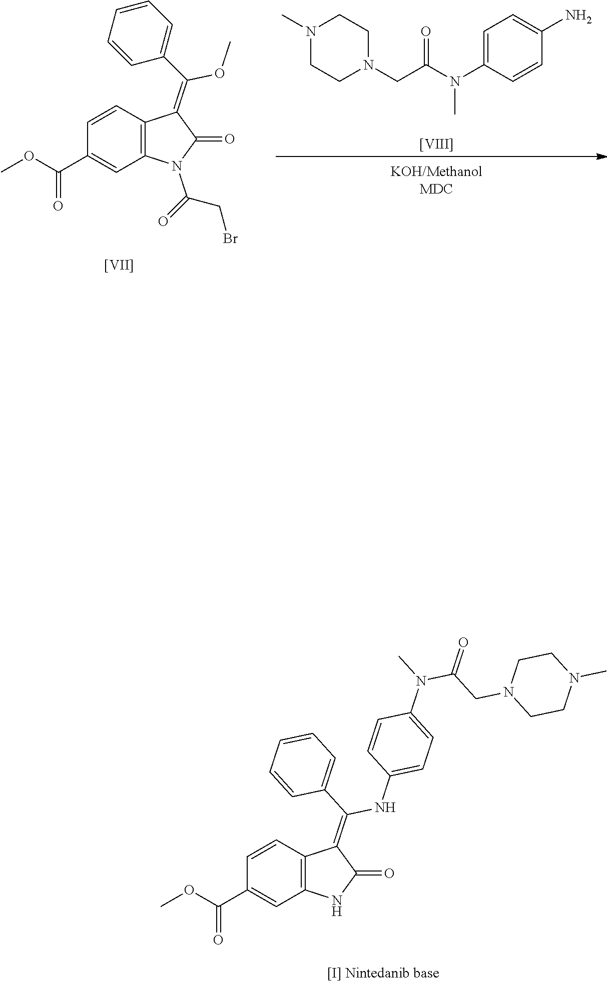

The present invention also provides salts of Nintedanib with acid selected from alkane sulfonic acid selected from ethane sulfonic acid preferably. Salts of Nintedanib as described by the present invention can be amorphous or crystalline.

One embodiment of the present invention includes preparation of Nintedanib salts which can be direct product of condensation reaction or can be alternatively prepared by the reaction of Nintedanib free base with a suitable acid.

According to another embodiment, present invention provides a process for the preparation of Nintedanib salt by reaction of Nintedanib free base with a suitable acid.

One embodiment of the present invention also includes a process for preparation of Nintedanib Esylate that involves reaction of Nintedanib in a suitable solvent with ethane sulfonic acid. In this process, Nintedanib free base, suitable solvent selected from methanol and purified water are charged into glass assembly at room temperature. The reaction mixture is stirred at 25-30° C. for 10-15 minutes and raise the temperature to 60-65° C. Then, alkane sulfonic acid selected preferably ethane sulfonic acid is added into reaction mass at 60-65° C. in 30 minutes. The reaction mass is stirred at 60-65° C. for 1.0 hr. The reaction mixture is filtered and wash with hot methanol and set temperature of reaction mass to 50-55° C. Then, isopropyl alcohol is added slowly to reaction mass at 50-55° C. in 20-30 minutes. Reaction mass is seeded with Nintedanib Esylate (Crystalline API) at 50-55° C. Again isopropyl alcohol is added into reaction mixture at 50-55° C. The reaction mixture is maintained at 50-55° C. for 30 minutes and then cooled to 0-5° C. and stir for 3 hr at same temperature. The precipitated solid is filtered and wash with isopropyl alcohol to get wet material. Dry the wet cake under vacuum at 45-50° C. to get methyl(3Z)-3-(((4-(n-methyl-2-(4-methylpiperazin-1-yl)acetamido)phenyl)amino) (phenyl)methylidene)-2-oxo-2,3-dihydro-1H-indole-6-carboxylate ethane sulfonate (Nintedanib Esylate) (II) as bright yellow solid.

After completion of salt formation, Nintedanib Esylate (II) can be isolated using suitable techniques such as filtration, centrifugation and the similar techniques.

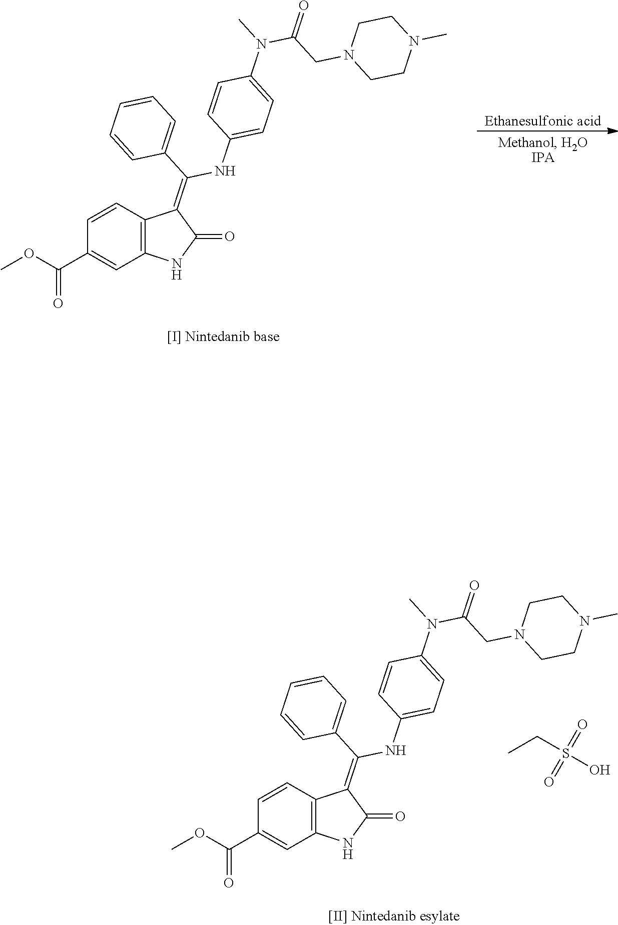

In another embodiment of the present invention relates to preparation of crystalline form BDR-NIN-1 of Nintedanib Esylate comprising addition of n-Heptane to crystallize out crystalline form BDR-NIN-1. This crystalline form has better physicochemical parameters than prior-art polymorphs which includes lower hygroscopic and good flow property.

In another embodiment of the present invention relates to preparation of crystalline form BDR-NIN-2 of Nintedanib Esylate comprising addition of Methyl ethyl ketone to crystallize out crystalline form BDR-NIN-2. This crystalline form also has better physicochemical parameters than prior-art polymorphs which includes lower hygroscopic and good flow property.

As per one embodiments of the present invention Nintedanib Esylate can be optionally purified to enhance purity and/or to remove impurity in the product. Any suitable purification method can be employed such as slurry wash, crystallization, base acid treatment and the like.

### EXAMPLES

Having described the invention with reference to certain preferred embodiments, other aspects will become apparent to one skilled in the art from consideration of the specification. The invention is further defined by reference to the following examples describing in detail by the preparation of the compounds of the invention. It will be apparent to those skilled in the art that many modifications, both to materials and methods, may be practiced without departing from the scope of the invention.

The following examples are provided for illustrative purpose only and these examples are in no way limitative on the present invention.

### Example-1: Preparation of methyl 1-(bromoacetyl)-2-oxo-2, 3-dihydro-1H-indole-6-carboxylate [V]

Suitable solvent toluene (300 ml) and methyl-2-oxo-2,3-dihydro-1H-indole-6-carboxylate (III) (100 gm) were charged into round-bottom flask at room temperature. The reaction mixture was stirred for 10-15 minutes to get homogeneous reaction mass. The reaction mass was heated to 105-110° C. Then, bromoacetyl bromide (IV) (211.16 gm) was added slowly into the reaction mass in 60 minutes at 105-110° C. The reaction mass was maintained for 8 hr at 105-110° C. Progress of reaction was monitored by TLC. Gradually, heating was stopped and the reaction mass was cooled to 60-65° C. The solvent was distilled out under vacuum below 65° C. completely. Toluene (200 ml) was added again into reaction mass and distilled out completely to remove traces of bromoacetyl bromide (IV). Toluene (300 ml) was added into reaction mass at 60-65° C. and the reaction mass was stirred for 10-15 minutes at same temperature. Then, n-heptane (200 ml) was added into reaction mass at 60-65° C. Gradually, the reaction mixture was cooled to 10-15° C. The reaction mixture was stirred for 3.0 hr at 10-15° C. The reaction mass was filtered and washed with mixture of Toluene and n-heptane (100 ml) (twice) to get titled wet material of compound [V] (173.5 gm).

### Example-2: Preparation of methyl (3Z)-1-(bromoacetyl)-3-[methoxy(phenyl)methylidene]-2-oxo-2,3-dihydro-1H-indole-6-carboxylate [VII]

In this, clean and dry glass assembly was set in downward distillation mode. Above prepared wet material (173.5 gm) of compound [V], toluene (775 ml) and acetic anhydride (177.83 gm) were charged into glass assembly at 25-30° C. The reaction mass was stirred for 10-15 minutes at 25-30° C. to get homogeneous reaction mass. The reaction mass was heated at 105-110° C. Then after, trimethyl orthobenzoate (317.12 gm) [VI] was added slowly into reaction mixture at 105-110° C. in 60 minutes. The reaction mass was maintained for 4 hr at same temperature. Volatile by product was removed gradually during reaction maintaining. Progress of reaction was monitored by TLC. The reaction mass was cooled to 55-60° C. Then, n-heptane (465 ml) was added into reaction mass at 55-60° C. The reaction mass was stirred for 15-20 minutes and then the reaction mixture was cooled to 10-15° C. and stirred for 2.0 hr at same temperature. The reaction mixture was filtered and washed with mixture of toluene and ethyl acetate (100 ml) (1:1). Obtained wet material was charged into another clean glass assembly. Methanol (400 ml) was added and reaction mass was stirred for 15 minutes at 25-30° C. The reaction mass was cooled at 0-5° C. and stirred for 2 hr at same temperature. Again, the reaction mass was filtered and washed with pre-chilled methanol (150 ml) to get wet material of compound (211.9 gm) [VII].

### Example-3: Preparation of Nintedanib Base [I]

Above prepared wet material (211.9 gm) of compound [VII] and methanol (795 ml) were charged into glass assembly. The reaction mass was heated to 60-65° C. KOH solution (8.56 gm in 100 ml methanol) was added slowly to reaction mass at 60-65° C. in 15-20 minutes. The reaction mass was maintained for 1 hr at 60-65° C. Progress of reaction was monitored by TLC. Then, methanol was distilled out completely under vacuum at below 60° C. Then, methanol (398 ml) was added again and stirred the reaction mass for 10-15 minutes at 60° C. Again methanol was distilled out completely under vacuum to remove traces of bromoacetic acid. Methanol (795 ml) was added to reaction mass at 60° C. N-(4-aminophenyl)-N-methyl-2-(4-methylpiperazin-1-yl)acetamide (133.25 gm) [VIII] was added into reaction mass at 60° C. The reaction mixture was maintained at 60-65° C. for 4 hr. Conversion was checked by TLC till completion of the reaction. Gradually heating was stopped and reaction mass was cooled to 10-15° C. Then, the reaction mass was cooled for 3 hr at 10-15° C. The precipitated solid was filtered and washed with pre-chilled (198 ml) methanol. Obtained wet material was charged again into glass assembly. Methanol (400 ml) was added into assembly at room temperature and the temperature was raised to 60-65° C. The reaction mass was stirred for 1 hr at 60-65° C. Then, the reaction mixture was cooled to 25-30° C. and stirred the reaction for 2 hr at same temperature. The reaction mass was filtered followed by washing with pre-chilled methanol (200 ml). Followed by unloading of wet cake and charged into another clean glass assembly. Methanol (600 ml) and dichloromethane (600 ml) were charged into assembly at room temperature to get clear solution. Then, activated charcoal (10.0 gm) was added into clear reaction mass and stirred for 1 hr at 25-30° C. The reaction mass was filtered through celite hyflo bed followed by washing with mixture of methanol and dichloromethane (1:1) (200 ml). Distilled out dichloromethane from filtrate ML at 40-55° C. Thenafter, methanol (400 ml) was added into reaction mass at 50-55° C. Gradually, the reaction mixture was cooled to 25-30° C. and stirred for 3.0 hr at same temperature. The reaction mass was filtered and washed with pre-chilled methanol (200 ml) to get wet material (267.9 gm). Followed by drying of the wet cake under vacuum at 45-50° C. to get pure Nintedanib base [I] (226.08 gm) as bright yellow solid. Total yield of pure Nintedanib base [I] was 80.14% (KSM to Base) and purity was 99.79%.

### Example-4: Preparation of Nintedanib Esylate [II]

Nintedanib free base [I] (200 gm), methanol (1134 ml) and purified water (16 ml) were charged into glass assembly at room temperature. Then, the reaction mixture was stirred at 25-30° C. for 10-15 and the temperature was raised to 60-65° C. Ethane sulfonic acid (41.22 gm) was added into the reaction mass at 60-65° C. in 30 minutes. The reaction mass was stirred at 60-65° C. for 1.0 hr. The reaction mixture was fine filtered using Whatmann filter paper at 60-65° C. and washed with hot methanol (200 ml). Remaining filtrate ML was transferred to another clean and dried glass assembly and set temperature of reaction mass to 50-55° C. Isopropyl alcohol (534 ml) was added slowly to reaction mass at 50-55° C. in 20-30 minutes. Reaction mass was seeded with Nintedanib Esylate (200 mg) (Crystalline API) at 50-55° C. Again isopropyl alcohol (800 ml) was added into reaction mixture at 50-55° C. Then, reaction mixture was maintained at 50-55° C. for 30 minutes. Gradually, the reaction mixture was cooled to 0-5° C. and stirred for 3 hr at same temperature. The precipitated solid was filtered and washed with isopropyl alcohol (200 ml) to get wet material (260.2 gm). Followed by drying of the wet cake under vacuum at 45-50° C. to get Nintedanib Esylate [II](232.1 gm) as bright yellow solid. Total yield of pure Nintedanib Esylate [II] was 96.22% and purity was 99.85%.

### Example-5: Preparation of Novel Crystalline Form BDR-NIN-1 from Nintedanib Esylate

Nintedanib Esylate was added in n-Heptane and dissolved slowly to reaction mass at 50-55° C. in 20-30 minutes. The solvent was evaporated to dryness to crystallize out novel crystalline form BDR-NIN-1. An XRD of BDR-NIN-1 is reported in figure-1 of this specification.

### Example-6: Preparation of Novel Crystalline Form BDR-NIN-2 from Nintedanib Esylate

Nintedanib Esylate was added in methyl ethyl ketone and dissolved slowly to reaction mass at 50-55° C. in 20-30 minutes. The solvent was evaporated to dryness to crystallize out novel crystalline form BDR-NIN-2. An XRD of BDR-NIN-2 is reported in figure-2 of this specification.

### Example-7: Stability Study

Nintedanib Esylate obtained as per the present invention was placed for long term stability study at 25° C. and 60% RH for six months. Results of Assay and impurities obtained are reported as below:

| | Time Interval | | | |
|---|---|---|---|---|
| Parameters | Initial | 1 Month | 3 Months | 6 Months |
| % Assay | 99.11 | 99.93 | 100.56 | 99.77 |

Impurities

| | | | | |
|---|---|---|---|---|
| Acid Impurity | Not Detected | 0.01% | 0.01% | 0.01% |
| Des-piprazinyl | Not Detected | 0.01% | 0.01% | 0.01% |
| Impurity | | | | |
| Piprazine Nintedanib | 0.08% | 0.07% | 0.09% | 0.08% |
| Dimer Impurity | | | | |
| Total Impurities | 0.22% | 0.25% | 0.22% | 0.25% |

Hence, from above data, it may be concluded that the inventors of the present invention have developed a robust invention by which a stable Nintedanib Esylate can be formed.

The invention described herein comprises in various objects and their description as mentioned above, with respect to characteristics and processes adopted. While these aspects are emphasised in the invention, any variations of the invention described above are not to be regarded as departure from the spirit and scope of the invention as described.

1. A process for the preparation of Nintedanib (I) comprising the steps of:
a) reacting methyl-2-oxo-2,3-dihydro-1H-indole-6-carboxylate (III) in the presence of a first organic solvent with bromoacetyl bromide (IV) to obtain methyl 1-(bromoacetyl)-2-oxo-2, 3-dihydro-1H-indole-6-carboxylate [V]; 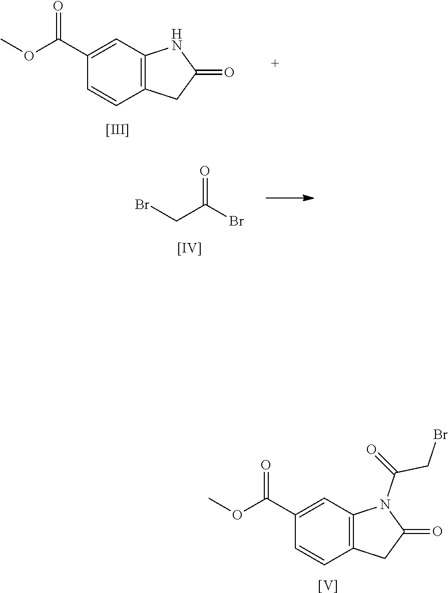
b) reacting the methyl 1-(bromoacetyl)-2-oxo-2, 3-dihydro-1H-indole-6-carboxylate [V] with trimethyl orthobenzoate [VI] in the presence of a second organic solvent to obtain methyl (3Z)-1-(bromoacetyl)-3-[methoxy(phenyl)methylidene]-2-oxo-2,3-dihydro-1H-indole-6-carboxylate [VII]; 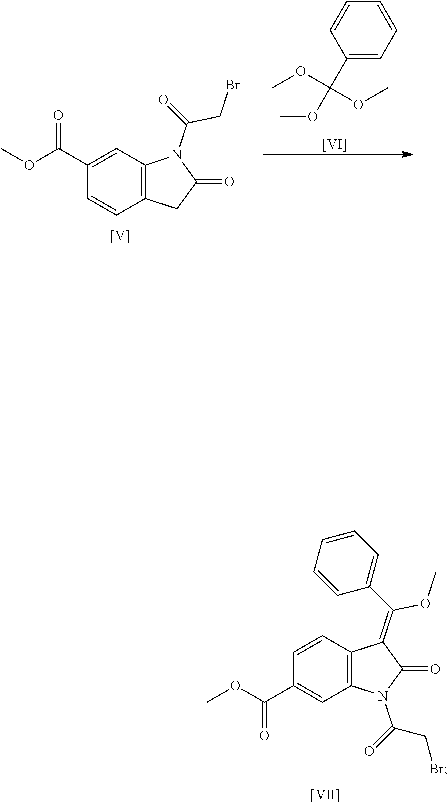
and
c) reacting the methyl (3Z)-1-(bromoacetyl)-3-[methoxy(phenyl)methylidene]-2-oxo-2,3-dihydro-1H-indole-6-carboxylate [VII] with N-(4-aminophenyl)-N-methyl-2-(4-methylpiperazin-1-yl)acetamide [VIII] in the presence of a third organic solvent to obtain Nintedanib (I):

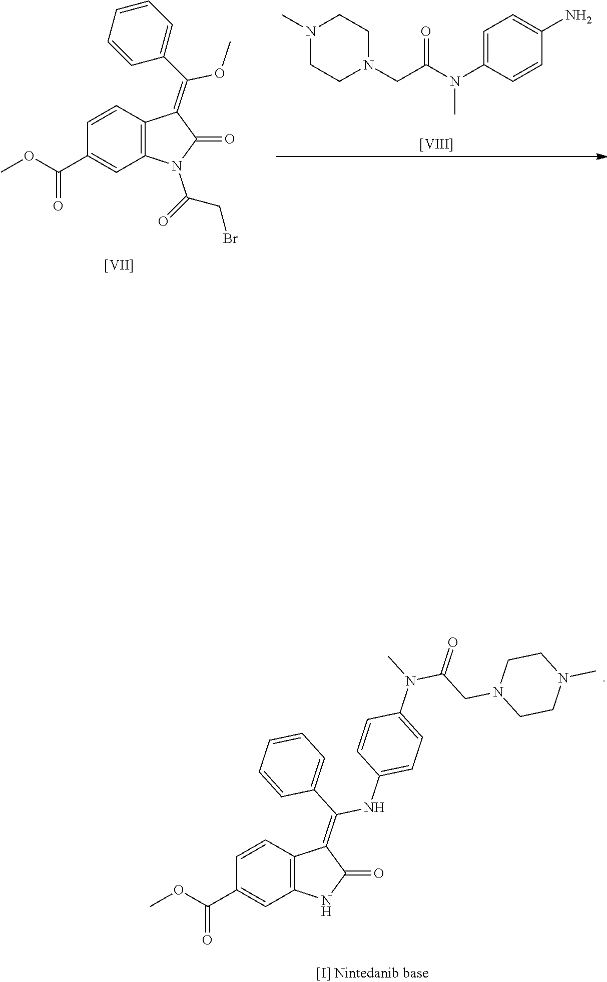

2. The process for the preparation of Nintedanib as claimed in claim 1, wherein the first organic solvent is selected from the group consisting of toluene, n-Heptane, and a mixture thereof.

3. A process for the preparation of Nintedanib Esylate comprising the steps of:
a) preparing Nintedanib (I) by the process of claim 1; and
b) reacting the Nintedanib (I) with ethanesulfonic acid in the presence of a fourth organic solvent and water to obtain Nintedanib Esylate.

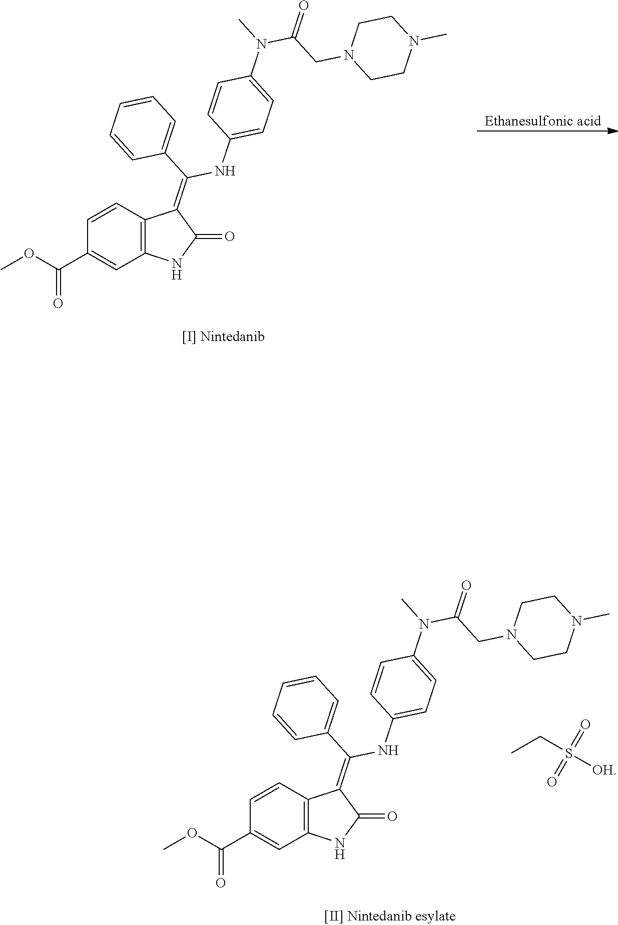

4. The process for the preparation of Nintedanib esylate as claimed in claim 3, wherein the fourth organic solvent is selected from the group consisting of methanol, isopropyl alcohol, and mixtures thereof.

5. (canceled)

6. (canceled)

7. A compound useful in the synthesis of nintedanib, wherein the compound is a compound of formula [V] or a compound of formula [VII]:

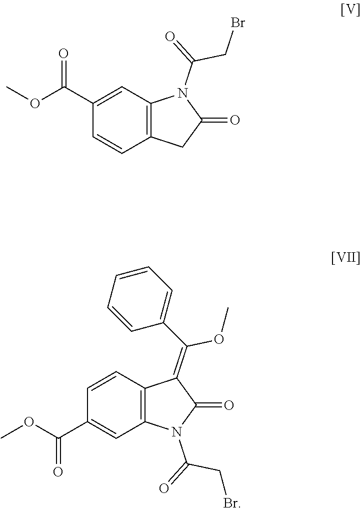 [SMILES: COC(=O)c1ccc2c(c1)N(C(=O)CBr)C(=O)C2 ; COC(=O)c1ccc2c(c1)N(C(=O)CBr)C(=O)/C2=C(\OC)c1ccccc1]

8. The intermediate of claim 7, wherein the intermediate is the compound of formula [VII]:

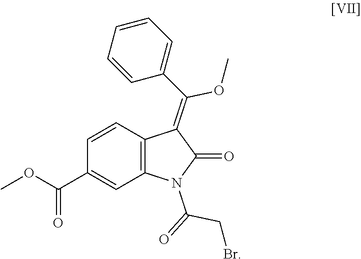 [SMILES: COC(=O)c1ccc2c(c1)N(C(=O)CBr)C(=O)/C2=C(\OC)c1ccccc1]

9. (canceled)

10. (canceled)

11. The improved process for the preparation of Nintedanib as claimed in claim 1, wherein the second organic solvent is selected from the group consisting of toluene, n-Heptane, acetic anhydride, methanol, and a mixture thereof.

12. The improved process for the preparation of Nintedanib as claimed in claim 1, wherein the third organic solvent is selected from the group consisting of methanol, dichloromethane, alcoholic potassium hydroxide, and a mixture thereof.

13. A process for the preparation of a novel nintedanib esylate crystalline form, comprising either:
dissolving nintedanib esylate in n-heptane to obtain an n-heptane solution; and crystallizing the nintedanib esylate from the n-heptane solution to obtain nintedanib esylate in crystalline form BDR-NIN-1, wherein crystalline form BDR-NIN-1 is characterized by an X-ray powder diffraction (XRD) pattern having major peaks at about 13.03, 17.80, 21.26 and 24.05±0.20 degrees 2-theta; or
dissolving nintedanib esylate in methyl ethyl ketone to obtain a methyl ethyl ketone solution; and crystallizing the nintedanib esylate from the methyl ethyl ketone solution to obtain nintedanib esylate in crystalline form BDR-NIN-2, wherein crystalline form BDR-NIN-2 is characterized by an XRD pattern having major peaks at about 6.54, 16.71, 18.81, 20.01, and 23.19±0.20 degrees 2-theta.

14. The process of claim 13, wherein the nintedanib esylate is dissolved in the n-heptane to obtain the n-heptane solution; and the nintedanib esylate is crystallized from the n-heptane solution to obtain nintedanib esylate in crystalline form BDR-NIN-1.

15. The process of claim 13, wherein the nintedanib esylate is dissolved in the methyl ethyl ketone to obtain the methyl ethyl ketone solution; and the nintedanib esylate is crystallized from the methyl ethyl ketone solution to obtain nintedanib esylate in crystalline form BDR-NIN-2.

16. The intermediate of claim 7, wherein the intermediate is the compound of formula [V]:

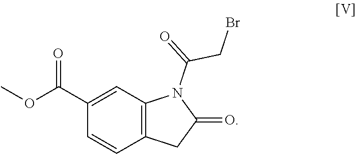 [SMILES: COC(=O)c1ccc2c(c1)N(C(=O)CBr)C(=O)C2]

17. A novel crystalline form of nintedanib esylate, wherein the crystalline form is either:
nintedanib esylate in crystalline form BDR-NIN-1, wherein the crystalline form BDR-NIN-is characterized by an X-ray powder diffraction (XRD) pattern having major peaks at about 13.03, 17.80, 21.26 and 24.05±0.20 degrees 2-theta; or
nintedanib esylate in crystalline form BDR-NIN-2, wherein the crystalline form BDR-NIN-is characterized by an XRD pattern having major peaks at about 6.54, 16.71, 18.81, 20.01, and 23.19±0.20 degrees 2-theta.

18. The novel crystalline form of claim 17, wherein the crystalline form is nintedanib esylate in crystalline form BDR-NIN-1.

19. The novel crystalline form of claim 17, wherein the crystalline form is nintedanib esylate in crystalline form BDR-NIN-2.

20. A process for the preparation of a novel nintedanib esylate crystalline form, comprising:
preparing nintedanib esylate by the process of claim 3; and
either:
dissolving the nintedanib esylate in n-heptane to obtain an n-heptane solution; and crystallizing the nintedanib esylate from the n-heptane solution to obtain nintedanib esylate in crystalline form BDR-NIN-1, wherein crystalline form BDR-NIN-1 is characterized by an X-ray powder diffraction (XRD) pattern having major peaks at about 13.03, 17.80, 21.26 and 24.05±0.20 degrees 2-theta; or
dissolving the nintedanib esylate in methyl ethyl ketone to obtain a methyl ethyl ketone solution; and crystallizing the nintedanib esylate from the methyl ethyl ketone solution to obtain nintedanib esylate in crystalline form BDR-NIN-2, wherein crystalline form BDR-NIN-2 is characterized by an XRD pattern having major peaks at about 6.54, 16.71, 18.81, 20.01, and 23.19±0.20 degrees 2-theta.
